(19) United States
(12) Patent Application Publication
(10) Pub. No.: US 2018/0055564 A1
(43) Pub. Date: Mar. 1, 2018

(54) SYSTEMS AND METHODS FOR NERVE DENERVATION TO RELIEVE PULMONARY DISEASE SYMPTOMS

(71) Applicant: Boston Scientific Scimed, Inc., Maple Grove, MN (US)

(72) Inventors: Bryan A. Clark, Forest Lake, MN (US); Aiden Flanagan, Caherweelder (IE)

(21) Appl. No.: 15/686,932

(22) Filed: Aug. 25, 2017

Related U.S. Application Data
(60) Provisional application No. 62/416,255, filed on Nov. 2, 2016.
(60) Provisional application No. 62/379,668, filed on Aug. 25, 2016.

(51) Int. Cl. A61B 18/14

(52) U.S. Cl. CPC A61B 18/1492; A61B 2018/00577

(57) ABSTRACT

The present disclosure relates to the field of denervation. Specifically, the present disclosure relates to systems and methods for preventing an electrical signal from travelling along a target nerve. In particular, the present disclosure relates to systems and methods for relieving a pulmonary symptom by denervating the internal branch of the superior laryngeal nerve.

### CROSS REFERENCE TO RELATED APPLICATIONS

The present application claims the benefit of priority under 35 U.S.C. §119 to U.S. Provisional Patent Application Ser. No. 62/416,255, filed on Nov. 2, 2016, and U.S. Provisional Patent Application Ser. No. 62/379,668, filed on Aug. 25, 2016, both of which are incorporated by reference in their entireties for all purposes.

### FIELD

The present disclosure relates to the field of denervation. Specifically, the present disclosure relates to systems and methods for effectively blocking an electrical signal from travelling along a target nerve. In particular, the present disclosure relates to systems and methods for relieving a pulmonary symptom by denervating a portion of the internal branch of the superior laryngeal nerve.

### BACKGROUND

Chronic obstructive pulmonary disease (COPD) includes conditions such as, e.g., chronic bronchitis and emphysema. COPD currently affects over 15 million people in the United States alone and is currently the third leading cause of death in the country. The primary cause of COPD is the inhalation of cigarette smoke, responsible for over 90% of COPD cases. The economic and social burden of the disease is substantial and is increasing.

Chronic bronchitis is characterized by chronic cough with sputum production. Due to airway inflammation, mucus hypersecretion, airway hyperresponsiveness, and eventual fibrosis of the airway walls, significant airflow and gas exchange limitations result.

Emphysema is characterized by the destruction of the lung parenchyma. This destruction of the lung parenchyma leads to a loss of elastic recoil and tethering which maintains airway patency. Because bronchioles are not supported by cartilage like the larger airways, they have little intrinsic support and therefore are susceptible to collapse when destruction of tethering occurs, particularly during exhalation.

Acute exacerbations of COPD (AECOPD) often require emergency care and inpatient hospital care. An AECOPD event is defined by a sudden worsening of symptoms (e.g., increase in or onset of cough, wheeze, and sputum changes) that typically last for several days, but can persist for weeks. An AECOPD event is typically triggered by a bacterial infection, viral infection, or pollutants, which manifest quickly into airway inflammation, mucus hypersecretion, and bronchoconstriction, causing significant airway restriction.

Despite relatively efficacious drugs (long-acting muscarinic antagonists, long-acting beta agonists, corticosteroids, and antibiotics) that treat COPD symptoms, a particular segment of patients known as “frequent exacerbators” often visit the emergency room and hospital with exacerbations and also have a more rapid decline in lung function, poorer quality of life, and a greater mortality risk.

Reversible obstructive pulmonary disease includes asthma and reversible aspects of COPD. Asthma is a disease in which bronchoconstriction, excessive mucus production, and inflammation and swelling of airways occur, causing widespread but variable airflow obstruction thereby making it difficult for the asthma sufferer to breathe. Asthma is further characterized by acute episodes of airway narrowing via contraction of hyper-responsive airway smooth muscle.

The reversible aspects of COPD include excessive mucus production and partial airway occlusion, airway narrowing secondary to smooth muscle contraction, and bronchial wall edema and inflation of the airways. Usually, there is a general increase in bulk (hypertrophy) of the large bronchi and chronic inflammatory changes in the small airways. Excessive amounts of mucus are found in the airways, and semisolid plugs of mucus may occlude some small bronchi. Also, the small airways are narrowed and show inflammatory changes.

In asthma, chronic inflammatory processes in the airway play a central role in increasing the resistance to airflow within the lungs. Many cells and cellular elements are involved in the inflammatory process including, but not limited to, mast cells, eosinophils, T lymphocytes, neutrophils, epithelial cells, and even airway smooth muscle itself. The reactions of these cells result in an associated increase in sensitivity and hyperresponsiveness of the airway smooth muscle cells lining the airways to particular stimuli.

The chronic nature of asthma can also lead to remodeling of the airway wall (i.e., structural changes such as airway wall thickening or chronic edema) that can further affect the function of the airway wall and influence airway hyper-responsiveness. Epithelial denudation exposes the underlying tissue to substances that would not normally otherwise contact the underlying tissue, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance, pharmacology and bronchial thermoplasty.

The autonomic nervous system (ANS) provides constant control over airway smooth muscle, secretory cells, and vasculature. The ANS is divided into two subsystems, the parasympathetic nervous system and the sympathetic nervous system. These two systems operate independently for some functions, and cooperatively for other functions. The parasympathetic system is responsible for the unconscious regulation of internal organs and glands. In particular, the parasympathetic system is responsible for sexual arousal, salivation, lacrimation, urination, and digestion, among other functions. The sympathetic nervous system is responsible for stimulating activities associated with the fight-or-flight response. Although both sympathetic and parasympathetic branches of the ANS innervate lung airways, it is the parasympathetic branch that dominates with respect to control of airway smooth muscle, bronchial blood flow, and mucus secretions.

FIG. 1 illustrates the cholinergic control of airway smooth muscle and submucosal glands. An airway 100 may include an inner surface 102 that includes epithelial tissue 104. Nerve fibers 106 may be C-fibers having a plurality of receptors 108 disposed within epithelial tissue 104. Nerve fibers 106 may be afferent (sensory) nerves that carry nerve impulses from receptors 108 toward central nervous system (CNS) 109. Receptors 108 may respond to a wide variety of chemical stimuli and other irritants, such as, e.g., cigarette smoke, histamine, bradykinin, capsaicin, allergens, and pollens. C-fibers can also be triggered by autocoids that are released upon damage to tissues of the lung. The stimulation of receptors 108 by the various stimuli elicits reflex cholinergic bronchoconstriction.

Parasympathetic innervation of the airways is carried by vagus nerve 110 (e.g., the right and left vagus nerves). Upon receiving a signal from nerve fiber 106, CNS 109 may send a signal to initiate bronchoconstriction and/or mucus secretion. Cholinergic nerve fibers (e.g., nerve fibers that use acetylcholine (ACh) as their neurotransmitter) arise in the nucleus ambiguous in the brain stem and travel down a vagus nerve 110 (right and left vagus nerves) and synapse in parasympathetic ganglia 112 which are located within the airway wall. These parasympathetic ganglia are most numerous in the trachea and mainstem bronchi, especially near the hilus and points of bifurcations, with fewer ganglia that are smaller in size dispersed in distal airways. From these ganglia, short post-ganglionic fibers 114 travel to airway smooth muscle 116 and submucosal glands 118. ACh, the parasympathetic neurotransmitter, is released from post-ganglionic fibers and acts upon M1- and M3-receptors on smooth muscles 116 and submucosal glands 118 to cause bronchoconstriction (via constriction of smooth muscles 116), and the secretion of mucus 122 within airway 100 by submucosal glands 118, respectively. ACh may additionally regulate airway inflammation and airway remodeling, and may contribute significantly to the pathophysiology of obstructive airway diseases. Thus, fibers 114 may be efferent fibers (motor or effector neurons) that are configured to carry nerve impulses away from CNS 109.

FIG. 2 illustrates additional afferent nerve fibers located in airway 100 and in airway smooth muscle 116. Airway 100 may include one or more nerve fibers 106 and receptors 108 as described with reference to FIG. 1. Additionally, one or more nerve fibers 206 having one or more receptors 208 may be disposed within epithelial tissue 104. Nerve fibers 206 may be myelinated Rapidly Adapting Receptors (RAR) that respond to mechanical stimuli and are responsible in part for bronchoconstriction. Receptors 208 may respond to mechanical stimuli such as, e.g., water, airborne particulates, mucus, and the stretching of the lung during breathing or coughing. RARs may cause bronchoconstriction and are triggered by merchant-stimulation (e.g., mechanical pressure or distortion) and/or chemo-stimulation. Additionally, RARs may be triggered secondary to bronchoconstriction, leading to an amplification of the constriction response.

Airway smooth muscle 116 may be coupled to one or more receptors 210. Receptors 210 may be, e.g., Slowly Adapting Receptors (SARs) that are coupled to one or more nerve fibers 211.

Bronchial hyperresponsivity (BHR) may be present in a considerable number of COPD patients. Various reports have suggested BHR to be present in between about 60% and 94% of COPD patients. This “hyperresponsivity” could be due to a “hyperreflexivity.” However, there are several logical mechanisms by which parasympathetic drive may be over-activated in inflammatory disease. First, inflammation is commonly associated with overt activation and increases in excitability of vagal C-fibers in the airways that could increase reflex parasympathetic tone. Secondly, airway inflammation and inflammatory mediators have been found to increase synaptic efficacy and decrease action potential accommodation in bronchial parasympathetic ganglia, effects that would likely reduce their filtering function and lead to prolonged excitation. Thirdly, airway inflammation has also been found to inhibit muscarinic M2 receptor-mediated auto-inhibition of ACh release from postganglionic nerve terminals. This would lead to a larger end-organ response (e.g., smooth muscle contraction) per a given amount of action potential discharge. Fourthly, airway inflammation has been associated with phenotypic changes in the parasympathetic nervous system that could affect the balance of cholinergic contractile versus non-adrenergic non-cholinergic (NANC) relaxant innervation of smooth muscle.

Because airway resistance varies inversely with the fourth power of the airway radius, BHR is believed to be a function of both bronchoconstriction and inflammation. Inflammation in the airway walls reduces the inner diameter (or radius) of the airway lumen, thus amplifying the effect of even baseline cholinergic tone, because for a given change in muscle contraction, the airway lumen will close to a greater extent. BHR is likely caused by hypersensitivity of receptor nerve fibers, such as, e.g., C-fibers, RAR fibers, SAR fibers, and the like, lower thresholds for reflex action initiation, and reduced self-limitation of acetylcholine release.

The majority of vagal afferent nerves in the lungs are nociceptors that are adept at sensing the type of tissue injury and inflammation that occurs in the lungs in COPD. In addition, stretch sensitive afferent nerves are present in the lungs and can be activated by the tissue distention that occurs during eupneic (normal) breathing. The pattern of action potential discharge in these fibers depends on the rate and depth of breathing, the lung volume at which respiration is occurring, and the compliance of the lungs. Therefore, because COPD patients exhibit impaired breathing, the activity of nociceptive and mechano-sensitive afferent nerves is grossly altered in patients with COPD. The distortion in vagal afferent nerve activity in COPD may lead to situations where these responses are out of sync with the body's needs.

There may be a clinical advantage for therapeutic treatments of the present disclosure to alleviate airway smooth muscle constriction, mucus production and other pulmonary symptoms before or during exacerbation events, such as acute exacerbations of COPD and/or asthma attacks, by effectively blocking signals from travelling along target nerves, such as vagal nerves.

### SUMMARY

The present disclosure, in its various aspects, meets an ongoing need in the medical field, such as the field of denervation, for systems and methods for permanently blocking an electrical signal from travelling along a target nerve. In particular, the present disclosure provides systems and methods for relieving a pulmonary symptom by permanently blocking an electrical signal from travelling along one or both internal branches of the superior laryngeal nerve (ib-SLN).

In one aspect, the present disclosure relates to a medical device comprising an expandable-collapsible element slidably disposed within a catheter lumen, wherein the expandable-collapsible element includes a framework formed from a plurality of longitudinal members, wherein the plurality of longitudinal members include regions of varying flexibility and stiffness configured to expand into a curved shape when not disposed within the catheter lumen, and one or more energy delivery elements carried about an outer surface of at least one of the longitudinal members. At least some of the longitudinal members may be connected to each other by one or more cross-pieces to form, e.g., a mesh-like structure or a basket structure. The longitudinal members may be joined together at a distal end of each longitudinal member. The energy delivery elements may be carried about the longitudinal members on only one side of the framework. The one or more energy delivery elements may include electrodes. The one or more energy delivery elements may be coupled to an ablation energy source. The one or more energy delivery elements may be coupled to an electrical activity processing system.

In another aspect, the present disclosure relates to a medical device comprising an expandable-collapsible element slidably disposed within a catheter lumen, wherein the expandable-collapsible element includes a balloon configured to move between a deflated configuration and an inflated configuration, and one or more energy delivery elements distributed along only one side of the balloon. The medical device may further include a vent tube passing through a center portion of the balloon. The one or more energy delivery elements may include electrodes. The one or more energy delivery elements may be coupled to an ablation energy source. The one or more energy delivery elements may be coupled to an electrical activity processing system. The electrodes may be configured to act as one or more of a sensing electrode, mapping electrode, stimulating electrode and/or ablation electrode.

In another aspect, the present disclosure relates to a multi-electrode assembly, comprising an elongate shaft that includes a proximal end, and a sharpened distal end, a plurality of deployable elements disposed about a distal portion of the elongate shaft, and one or more electrodes disposed along a length of each of the deployable elements. Each of the electrodes may be coupled to an ablation energy source. Each of the electrodes may be coupled to an electrical activity processing system. Each of the electrodes may be configured to act as one or more of a sensing electrode, mapping electrode, stimulating electrode and/or ablation electrode.

In another aspect, the present disclosure relates to a method, comprising introducing an expandable-collapsible element into a body lumen, wherein the expandable-collapsible element includes a framework formed from a plurality of longitudinal members, wherein the plurality of longitudinal members include regions of varying flexibility and stiffness configured to expand into a curved shape when not disposed within the catheter lumen, and one or more energy delivery elements carried about an outer surface of at least one of the longitudinal members; moving the expandable-collapsible element to an expanded configuration such that one or more of the energy delivery elements contact a tissue of the body lumen; and delivering ablation energy from at least one of the energy delivery elements to the tissue of the body lumen. The energy delivery elements may be carried about the longitudinal members on only one side of the framework. At least some of the longitudinal members are connected to each other by one or more cross-pieces to form, e.g., a mesh-like structure or a basket structure. Alternatively, the expandable-collapsible element may include a balloon configured to move between a deflated configuration and an inflated configuration. The energy delivery elements may be distributed along only one side of the balloon.

### BRIEF DESCRIPTION OF THE DRAWINGS

Non-limiting examples of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of skill in the art to understand the disclosure. In the figures:

FIG. 1 is a schematic view of an airway and a cholinergic pathway.

FIG. 2 is a schematic view of an airway and afferent nerves.

FIGS. 3A-3D illustrate electrodes carried on an outer surface of a collapsible-expandable framework, according to an embodiment of the present disclosure.

FIGS. 4A-4B illustrate fluid delivery ports disposed along an outer surface of a collapsible-expandable framework, according to an embodiment of the present disclosure.

FIGS. 5A-5B illustrate electrodes carried on an outer surface of a collapsible-expandable framework, according to an embodiment of the present disclosure.

FIG. 6 illustrates a collapsible-expandable framework within the larynx of a patient, according to an embodiment of the present disclosure.

FIG. 7 illustrates a multi-electrode assembly disposed about the internal branch of the superior laryngeal nerve, according to an embodiment of the present disclosure.

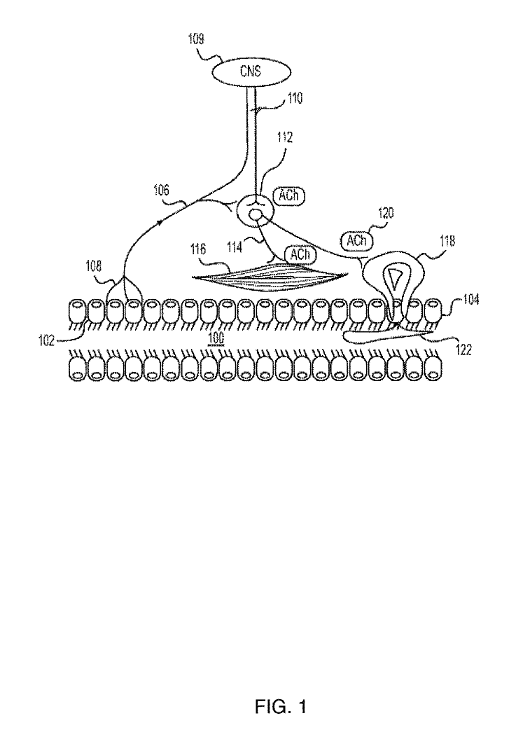

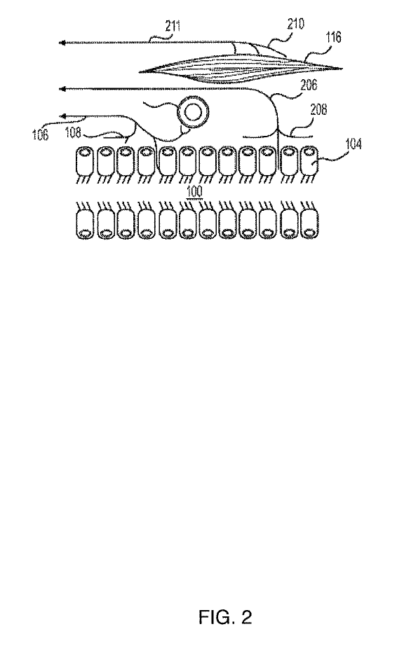

It is noted that the drawings are intended to depict only typical or exemplary embodiments of the disclosure. Accordingly, the drawings should not be considered as limiting the scope of the disclosure. The disclosure will now be described in greater detail with reference to the accompanying drawings.

### DETAILED DESCRIPTION

Before the present disclosure is described in further detail, it is to be understood that the disclosure is not limited to the particular embodiments described, as such may vary. It is also to be understood that the terminology used herein is for the purpose of describing particular embodiments only, and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure are described with specific reference to systems and methods for denervating one or both internal branches of the superior laryngeal nerve to relieve pulmonary symptoms, it should be appreciated that such systems and methods may be used to denervate a variety of nerves throughout and along locations in the nervous system to diagnose and treat a variety of acute or chronic symptoms.

As used herein, the term “distal” refers to the end farthest away from a medical professional when introducing a device into a patient, while the term “proximal” refers to the end closest to the medical professional when introducing a device into a patient.

As used herein, an “expandable,” “expanded,” “inflated” configuration and grammatical equivalents thereof refers to a state of an increased diameter as compared to a state of decreased diameter, such as in a “collapsible,” “collapsed,” “non-expanded” or “deflated” configuration. As used herein, “diameter” refers to the distance of a straight line extending between two points and does not necessarily indicate a particular shape.

The systems and methods of the present disclosure are described herein with particular exemplary reference to relieving pulmonary systems (e.g., asthma, COPD, airway smooth muscle contraction (ASM), mucus production chronic coughing, dyspnea, dynamic hyperinflation, etc.) by denervating one or both internal branches of the superior laryngeal nerve (ib-SLN). It should be appreciated that the ib-SLN protects the respiratory tract by mobilizing the glottis closure reflex during swallowing, coughing and vomiting. For this reason, bilateral denervation of the ib-SLN may lead to phonation disorders and disorders of respiratory control. To avoid such problems, the systems disclosed herein may be configured to only denervate one branch of the ib-SLN.

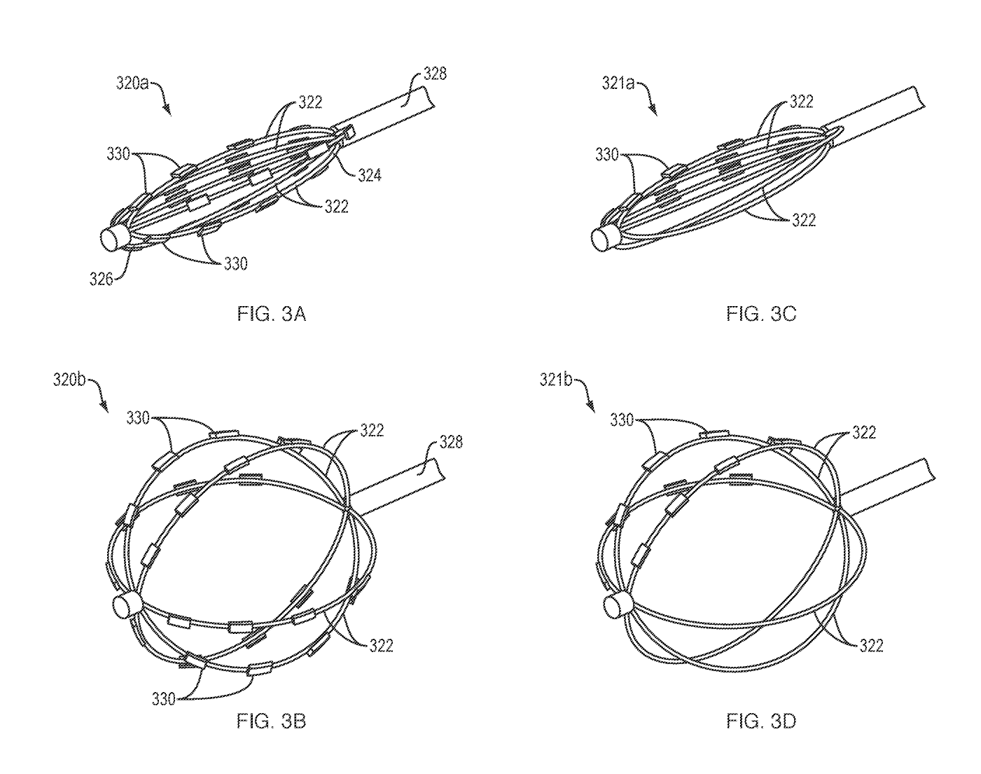

Referring to FIGS. 3A-3B, in one embodiment, the present disclosure provides an expandable-collapsible framework configured to move between a collapsed configuration 320a (FIG. 3A) and an expanded configuration 320b (FIG. 3B). When in the collapsed configuration 320a, the framework may be slidably disposed within the lumen of a catheter (not depicted) for delivery into a body lumen, including, for example, the larynx. The framework may include a proximal end 324 and a distal end 326, between which a plurality of flexible curved longitudinal members 322 (e.g., flexible spines) extend in a circumferentially spaced relationship. The proximal end 324 of the framework may be coupled to a delivery wire 328. In some embodiments, the longitudinal members 322 that form the framework may include regions of varying flexibility and/or stiffness such that the framework conforms to various larynx shapes and sizes when in the expanded configuration. In one embodiment, adjacent longitudinal members 322 may be connected by a series of cross-pieces (not depicted) to form a mesh- or basket-like structure with enhanced structural integrity that defines an interior space when in the expanded configuration. A plurality of unipolar, bipolar or multipolar electrodes 530 may adhered, bonded or otherwise affixed to an outer surface 546 of the basket-like structure. The longitudinal members 322 and struts may be made, for example, from a resilient inert material, including metals and metal alloys such as platinum, tungsten, titanium, stainless steel, nickel and nickel-titanium alloys (e.g., nitinol), polymers such as acrylate-based polymers, polyurethane-based polymers, polynorbornene-based polymers, and polylactide-based polymers, and any combinations thereof.

A plurality of unipolar, bipolar or multipolar electrodes 330 may be disposed both around and along the outer surface of the longitudinal members 322. For example, the framework may include six longitudinal members 322, with four electrodes 330 disposed along the length of each longitudinal member. It should be appreciated that any number of longitudinal members and/or electrodes may be disposed about the outer surface of the framework to electrically ablate and/or map the tissues of the laryngeal wall. Although the electrodes are depicted as protruding outward from the longitudinal member(s), it should be appreciated that the electrodes may include various configurations, including low-profile isodiametric arrangement along the longitudinal member(s). For example, there may be one, two, three, four, five, six, seven, eight, nine, ten, eleven, twelve, sixteen, twenty, twenty-four, or more electrodes. Alternatively, the electrodes may be disposed along some of the spines framework, while other spines do not carry any electrodes. For example, as illustrated in FIGS. 3C-3D, the expandable-collapsible framework 321a, 321b may include three longitudinal members 322 with electrodes 330 disposed along a length thereof, and three longitudinal members 322 on an opposite side of the framework that do not carry any electrodes.

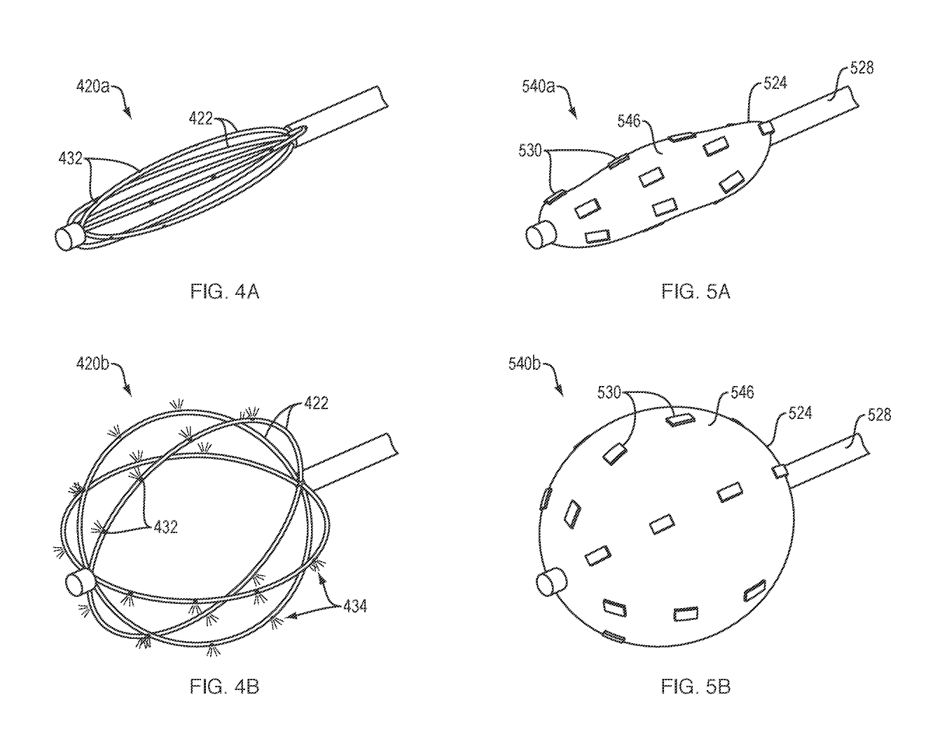

Referring to FIGS. 4A-4B, in one embodiment, the framework 420a, 420b may include a plurality of fluid-delivery ports 432 disposed both around and along the outer surface of each longitudinal member 422. For example, the framework may include six longitudinal members 422, with four fluid-delivery ports 432 disposed along the length of each longitudinal member. Alternatively, the fluid-delivery ports 432 may be disposed along some of the longitudinal members 422, while other longitudinal members 422 do not carry any fluid delivery ports. For example, the framework may include three longitudinal members 422 with fluid-delivery ports 432 disposed along a length thereof, and three longitudinal members 422 on an opposite side of the framework that do not carry any fluid delivery ports. Although not depicted, it should be appreciated that the framework of FIGS. 4A-4B may also include a plurality of electrodes, e.g., disposed adjacent to each of the fluid delivery ports.

Each longitudinal member 422 of the framework may include a lumen fluidly connected to an external source of ablation fluid (not shown). An ablative liquid (e.g., capsaicin, ethanol etc.) or gas (e.g., liquid nitrogen) that sufficiently affects nerve activity to effectively block nerve signal transmission may be delivered under pressure from the external fluid source through the lumen of each longitudinal member such that a spray 434 of the ablative liquid or gas is emitted from each fluid-delivery port. In one embodiment, each longitudinal member 422 may be fluidly connected to the external fluid source by a separate fluid delivery lumen, such that ablative liquid may be selectively emitted from some of the fluid-delivery ports 432. For example, ablative liquid may be emitted from the fluid-delivery ports located on one side of the framework, while the fluid delivery ports on the opposite side of the framework do not emit any ablative liquid.

Referring to FIGS. 5A-5B, in one embodiment, the present disclosure provides a balloon as a collapsible-expandable framework configured to move between a deflated or non-expanded configuration 540a (FIG. 5A) and an inflated or expanded configuration 540b (FIG. 5B). When in the deflated configuration 540a, the balloon may be slidably disposed within the lumen of a catheter (not depicted) for delivery into a body lumen, including, for example, a larynx, trachea or esophagus. A proximal end 524 of the balloon may be attached to a delivery shaft 528 which defines a lumen configured to deliver and/or remove an inflation fluid (e.g., a biologically inert liquid, gel or gas) between a fluid source (not depicted) and an interior of the balloon. A plurality of unipolar, bipolar or multipolar electrodes 530 may adhered, bonded or otherwise affixed to an outer surface 546 of the balloon. Any number of electrodes in any number of patterns may be disposed about the outer surface of the balloon to electrically ablate and/or map the tissues of the laryngeal wall. For example, there may be one, two, three, four, five, six, seven, eight, nine, ten, eleven, twelve, sixteen, twenty, twenty-four, or more electrodes. To ensure that the ablation energy is delivered to the appropriate portion of the laryngeal wall, the electrodes may be uniformly disposed about the outer surface of the balloon. The electrodes may also be disposed non-uniformly, for example, with greater density at areas adjacent to the ib-SLN and lower density at the top and/or bottom portions of the balloon that do not substantially contact the laryngeal wall. Alternatively, electrodes may be distributed along only one side of the balloon such that an opposite side of the balloon does not include any electrodes. In one embodiment, the balloon may include a profile when in the inflated or expanded configuration that allows the continuous flow (i.e., circulation) of air to the lungs during treatment. For example, the balloon may include a vent tube that passes through a center portion of the balloon. In addition, or alternatively, the balloon may include a profile (e.g., star shape, X-shape, etc.) that allows a portion of the balloon wall to be placed in contact with the laryngeal wall, while another portion of the balloon wall does not contact the laryngeal wall.

The balloon depicted in FIGS. 5A-5B may be formed of a flexible material such that it conforms to the shape of the larynx to engage each or substantially each electrode or a subset of electrodes against the laryngeal wall. The non-compliant nature of the balloon ensures that the balloon does not over-expand, thereby maintaining firm contact with the laryngeal wall. As will be understood by those of skill in the art, a balloon can be formed using any suitable technique, such as blow molding, film molding, injection molding, dip coating and/or extrusion, among others. For example, a polymer tube can be extruded, and can thereafter stretched and blown to form a balloon. Methods of forming a balloon from a polymer tube are described, for example, in commonly-assigned U.S. Ser. No. 10/263,225, filed Oct. 2, 2002, and entitled “Medical Balloon;” Anderson, U.S. Pat. No. 6,120,364; Wang, U.S. Pat. No. 5,714,110; and Noddin, U.S. Pat. No. 4,963,313, all incorporated herein by reference in their entirety.

The balloon may include a combination of elastomeric and non-compliant materials. For example, balloon can include one or more thermoplastics and/or thermosets. Examples of thermoplastics include polyolefins; polyamides (e.g., nylon, such as nylon 12, nylon 11, nylon 6/12, nylon 6, nylon 66); polyesters (e.g., polyethylene terephthalate (PET), polybutylene terephthalate (PBT), polyethylene naphthalate (PEN), polytrimethylene terephthalate (PTT)); polyethers; polyurethanes; polyvinyls; polyacrylics; fluoropolymers; copolymers and block copolymers thereof, such as block copolymers of polyether and polyamide (e.g., PEBAX®); and mixtures thereof. Examples of thermosets include elastomers (e.g., EPDM), epichlorohydrin, polyureas, nitrile butadiene elastomers and silicones. Other examples of thermosets include epoxies and isocyanates. Biocompatible thermosets may also be used. Biocompatible thermosets include, for example, biodegradable polycaprolactone, poly(dimethylsiloxane) containing polyurethanes and ureas and polysiloxanes. Ultraviolet curable polymers, such as polyimides and acrylic or methacrylic polymers and copolymers can also be used. Other examples of polymers that can be used in balloons include polyethylenes, polyethylene ionomers, polyethylene copolymers, polyetheretherketone (PEEK), thermoplastic polyester elastomers (e.g., Hytrel®) and combinations thereof. The balloon may include multiple layers provided by, for example, co-extrusion. Other polymers are described, for example, in U.S. Pat. Pub. No. 2005/0043679, filed on Aug. 21, 2003, entitled “Medical Balloons,” which is incorporated herein by reference in its entirety.

Because there is significant variation in larynx size and shape between individuals, a compliant balloon material is desirable in some embodiments in order to contact various surfaces. Unlike non-compliant balloons, compliant balloons are composed of materials that do not expand to a single pre-determined volume, but may preferably expand to a volume in the range of 10% to 800% and more, and more preferably in the range of 50% to 200% compared to a non-compliant balloon with a similar uninflated volume. Examples of compliant balloon materials include elastomers such as silicone rubber, ethylene-propylene-diene copolymers, butyl rubber, styrene-isobutylene-styrene copolymers, urethanes, and latexes, among others.

In some embodiments, the distal end of the catheter may include a steerable segment comprising a flexible and/or bendable material. Examples of steerable catheters may be found, for example, in U.S. Pat. Nos. 5,656,030 and 6,837,867 and U.S. Patent Publication No. 20100010437, the entire disclosures of which are hereby incorporated by reference. The steerable segment allows the framework or balloon to be precisely positioned within the larynx to ensure that the spines or balloon surface are placed direct apposition with the portion of the laryngeal wall adjacent to the ib-SLN.

In additional aspects, members 320a, 320b, 420a, 420b or balloon 540a, 540b, may include color-coded markings for visualization (e.g., using a bronchoscope or laryngoscope) to allow the physician to verify that the electrodes are properly positioned within the larynx prior to initiating the mapping and/or ablation steps (discussed below). In addition, or alternatively, the longitudinal members 322, 422 or balloon may incorporate radiopaque markers along their length to allow the physician to verify their position within the larynx using, for example, fluoroscopy or X-ray imaging.

Each electrode may be electrically coupled to an electrical activity processing system and source of electrical energy, such as ablative energy (e.g., radiofrequency energy) by a separate electrically conductive wire or wires that extends along an inner or outer surface of the framework (FIGS. 3A-3D) or balloon (FIGS. 5A-5B). In another embodiment, the electrodes may be electrically coupled to an electrical activity processing system and an ablation energy source by one or more flexible circuits printed along the inner or outer surface of the spines of the members or balloon surface. In yet another embodiment, the flexible printed circuits may form the spines of the framework. Such electrodes and conductive wires may be formed from materials commonly used in cardiac, denervation, neurostimulation and other medical electrodes and catheters, including suitable insulative materials, e.g., ETFE, PTFE, silicone, and PU and conductive materials, e.g., MP35N, stainless steel, Pt—Ir, Nitinol, Elgiloy and the like. The electrodes may be controlled in a number of ways, e.g., individually, in series, in parallel, in groups, spine-by-spine, by row or column.

In one embodiment, the electrical activity processing system may be configured to measure (e.g., map) the intrinsic electrical activity of a target nerve that innervates the tissues of the laryngeal wall. Examples of mapping electrodes for use with medical ablation systems may be found, for example, in U.S. Patent Publication Nos. 2008/0249518 and 2002/0177765, each of which are hereby incorporated by reference in their entirety. As mapping electrodes, the conductive wire connected to each electrode may be electrically coupled to the input of an electrical activity processing system (not shown), such as, for example, an electromyograph. Each electrode may be assigned an electrode location and an electrode channel within the electrical activity processing system. The electrical activity processing system may be configured to detect the intrinsic electrical activity of the target nerve (e.g., ib-SLN) which innervates the tissues of the laryngeal wall. For example, intrinsic nerve activity may be mapped by delivering low frequency electrical stimulation to the laryngeal wall, and detecting/monitoring the action potential(s) elicited from the target nerve(s). The electrical activity processing system may then process the action potentials to identify which electrode(s) should be energized to ablate the target nerve. The duration and/or intensity of the ablation energy may vary as necessary to effectively block the target nerve activity.

In one embodiment, following delivery of the ablation energy, the mapping function of the electrodes may be re-established to determine the amount, if any, of residual electrical activity emitted along the target nerve. In the event that residual electrical activity is detected, the previously identified electrodes may be re-energized to deliver additional ablation energy to the target nerve. This process may be repeated as necessary until the electrical activity of the target nerve is effectively blocked. The ability of the electrodes to repeatedly monitor and ablate target regions of the laryngeal wall ensures that ablation energy is delivered directly to the target nerve without prolonging the duration or intensity of the energy. This targeted approach not only focuses the ablation energy on the selected regions of the laryngeal wall, but minimizes or eliminates unwanted and potentially harmful ablation of surrounding tissues and non-target nerves.

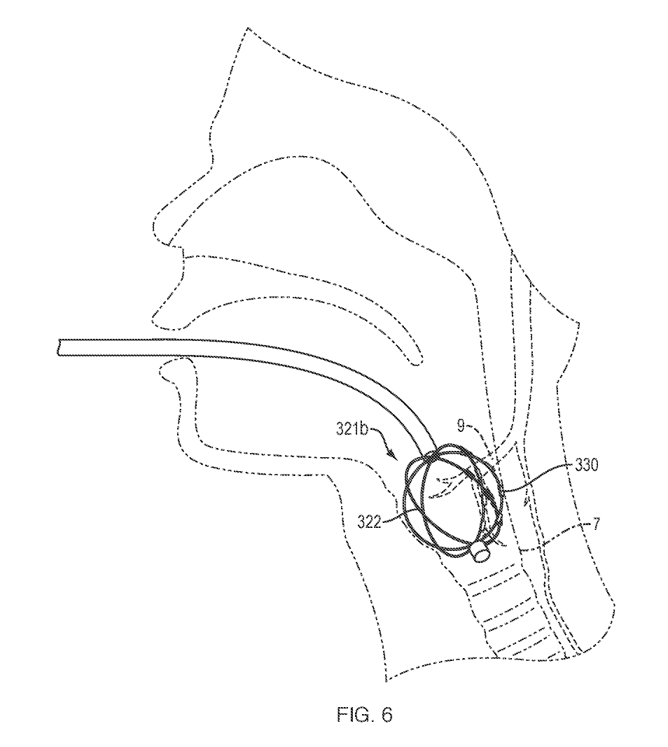

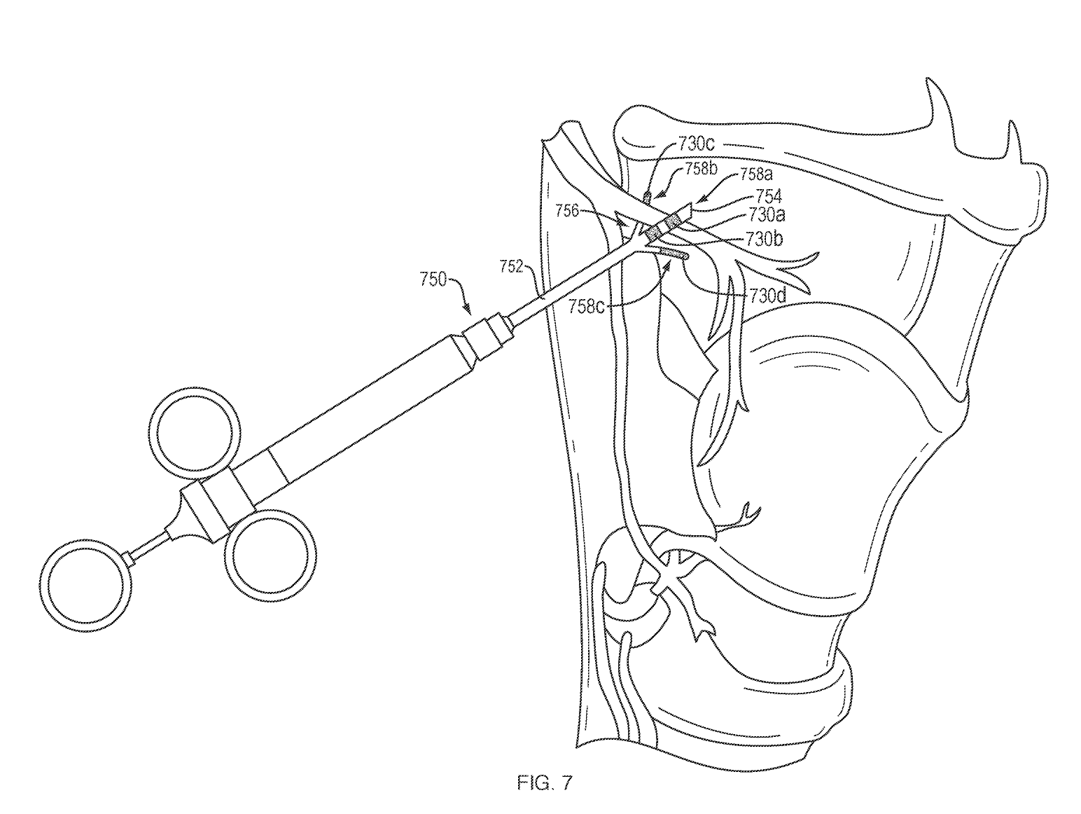

In use and by way of example, the framework may be slidably disposed within the lumen of a catheter configured to be inserted into the larynx of the patient. In various embodiments, when in the collapsed configuration the framework may have a profile (i.e., diameter) less than 3.7 mm, preferably less than 2.8 mm, and more preferably less than or equal to 2.5 mm. It will be appreciated that the framework may be provided in a variety of different collapsed and expanded dimensions in order to treat a range of larynx sizes. Once the catheter is properly positioned within the larynx 7 (FIG. 6) adjacent to the ib-SLN 9, the framework may be advanced beyond the distal end of the catheter such that the framework moves from the collapsed configuration to an expanded configuration 321b, thereby placing at least some of the electrodes 330 (or fluid-delivery ports 432) on the outer surface of the longitudinal member(s) 322, 422 into contact with the tissues of the laryngeal wall (FIG. 6). The framework may move from the collapsed configuration to an expanded configuration using, e.g., a proximally retracting a pull wire attached to a distal end of the framework.

Once the framework is properly positioned within the larynx, the mapping function may be initiated to identify which electrode(s) are in contact with a tissue of the laryngeal wall that is adjacent to and/or innervated by the ib-SLN. Ablation energy may then be selectively delivered to those identified electrodes to ablate the ib-SLN. Alternatively, the mapping function may be skipped entirely, and ablation energy delivered to those electrodes which the medical professional identifies as being in sufficiently close proximity to the ib-SLN (e.g., the region of the larynx between the glottis and laryngeal vestibule). The framework may then be returned to the collapsed configuration within the lumen of the endoscope and retracted from the larynx.

Alternatively, the balloon of FIGS. 5A-5B may be slidably disposed within the lumen of a catheter configured to be inserted into the larynx of the patient. Once the catheter is properly positioned within the larynx 7 adjacent to the ib-SLN 9, the balloon may be advanced beyond the distal end of the catheter and moved from the deflated configuration 540a to the inflated configuration 540b by flowing an inflation fluid from a fluid source (not depicted) into an interior of the balloon. In various embodiments, when in the deflated configuration 540a the balloon may have a profile (i.e., diameter) less than 3.7 mm, preferably less than 2.8 mm, and more preferably less than or equal to 2.5 mm. It will be appreciated that the balloon may be provided in a variety of different deflated and inflated dimensions in order to treat a range of larynx sizes. The balloon may be expanded such that it conforms to the inner dimensions of the larynx 5, thereby placing at least some of the electrodes 530 on the outer surface 546 of the balloon into contact with the tissues of the laryngeal wall. Continued exertion of pressure from the inflation fluid maintains the balloon in the expanded configuration 540b such that the electrode(s) 530 remain in contact with the tissues of the laryngeal wall. In one embodiment, the inflation fluid may be continuously or intermittently circulated through the balloon to maintain the balloon in its expanded configuration. In addition, circulating inflation fluid may serve as a coolant to maintain an appropriate temperature of tissues in direct contact with, or in the vicinity of, the balloon surface.

Once the balloon is properly positioned within the larynx, the mapping function may be initiated to identify which electrode(s) are in contact with a tissue of the laryngeal wall that is adjacent to and/or innervated by the ib-SLN. Ablation energy may then be selectively delivered to those identified electrodes to ablate the ib-SLN. Alternatively, the mapping function may be skipped entirely, and ablation energy delivered to those electrodes which the medical professional identifies as being in sufficiently close proximity to the ib-SLN. The balloon may then be returned to the deflated configuration and retracted from the larynx.

Referring to FIG. 7, in one embodiment, the present disclosure provides a multi-electrode assembly 750 configured to be advanced through the patient's skin to deliver denervation energy directly to the ib-SLN 9. The multi-electrode assembly 750 may include an elongate shaft 752 with deployable prongs 758a-c configured to move from a first delivery configuration to a second deployed configuration. Although deployable prong 758a is depicted as including a pointed/sharpened distal end 754, in various embodiments, any or all of the deployable prongs may include a pointed/sharpened distal end. One or more unipolar, bipolar or multipolar electrodes 730a-d may be disposed on an inner and/or outer surface of each deployable prong 758a-c. As above, each electrode 730a-d may be electrically coupled to an electrical activity processing system and an ablation energy source by a separate electrically conductive wire or wires (not depicted) that extend along an inner or outer surface of the deployable prongs 758a-c and elongate shaft 752. The electrical activity processing system may be configured to identify the intrinsic electrical activity of a target nerve 9 (e.g., ib-SLN) disposed between two or more of the deployable prongs 758a-c.

Each conductive wire may also be electrically coupled to an energy source (not shown) configured to selectively deliver ablation energy between the deployable prongs 758a-c identified as being on either side of the target nerve (i.e., ib-SLN). For example, as illustrated in FIG. 7, ablative energy may be delivered between electrodes 730a and 730b on a first deployable prong 758a and electrode 730c of a second deployable prong 758b to ablate a portion of the ib-SLN 9 lying between the first and second deployable prongs 758a, 758b. By comparison, ablative energy may not be delivered between electrode 730d of third deployable prong 758c and any of electrodes 730a, 730b and/or 730c because the target nerve is not disposed therebetween. The duration and/or intensity of the ablation energy may vary as necessary to effectively block electrical activity of the target nerve, as discussed above. For example, ablation energy may be provided as a pulse, or series of pulses, of radio frequency (RF) energy. In one embodiment, the pulsed RF energy delivered between the deployable prongs may effect denervation of the target nerve. In another embodiment, the multi-electrode assembly 750 may include a temperature sensor configured to measure the temperature of the target nerve and/or surrounding tissue and prevent the pulsed RF energy from exceeding a threshold temperature, e.g., approximately 45-65 degrees Celsius. The mapping function of the electrodes may then be re-established to determine if the ablation of the target nerve 9 has been achieved. In the event that the electrical activity within the target nerve persists, the respective prongs may be re-energized with ablation energy. This process may be repeated as necessary to effectively block the electrical activity of the target nerve. The ability of these electrode arrays to repeatedly monitor and ablate specific portions of the target nerve ensures that focal energy is delivered only to the target region, and without prolonging the duration or intensity of the energy. This targeted approach not only focuses the energy on the selected regions in need of ablation, but minimizes or eliminates unwanted and potentially harmful ablation of surrounding healthy/normal tissue.

It should be appreciated that ablation energy of the present disclosure is not limited to RF energy, but may include a variety energy sources, including, for example, irreversible electroporation (IRE energy), microwave electromagnetic energy, laser energy, acoustic energy and/or chemical energy, among others. For example, the energy source may include a conventional RF power supply that operates at a frequency in the range from 200 KHz to 1.25 MHz, with a conventional sinusoidal or non-sinusoidal wave form. Suitable power supplies are capable of supplying an ablation current at a relatively low voltage, typically below 150V (peak-to-peak), usually being from 50V to 100V. Power supplies capable of operating within these ranges are available from commercial vendors. Alternatively, the ablative energy may include an ablative liquid (e.g., capsaicin, ethanol or other suitable chemicals) or gas (e.g., liquid nitrogen) emitted from the fluid-delivery port(s) which are in contact with a tissue of the laryngeal wall that is adjacent to and/or innervated by the ib-SLN. For example, a plurality of microneedles may extend outward from an outer surface of one or more of the longitudinal members of the framework, or the outer surface of the balloon, to provide slight penetration of the target tissue for delivery of the ablative liquid.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
an expandable-collapsible element slidably disposed within a catheter lumen, wherein the expandable-collapsible element includes a framework formed from a plurality of longitudinal members, wherein the plurality of longitudinal members include regions of varying flexibility and stiffness configured to expand into a curved shape when not disposed within the catheter lumen; and
one or more energy delivery elements carried about an outer surface of at least one of the longitudinal members.

2. The medical device of claim 1, wherein at least some of the longitudinal members are connected to each other by one or more cross-pieces.

3. The medical device of claim 2, wherein the longitudinal members and one or more cross-pieces form a mesh-like structure.

4. The medical device of claim 2, wherein the longitudinal members and one or more cross-pieces form a basket structure.

5. The medical device of claim 1, wherein the longitudinal members are joined together at a distal end of each longitudinal member.

6. The medical device of claim 1, wherein the energy delivery elements are carried about the longitudinal members on only one side of the framework.

7. The medical device of claim 1, wherein the one or more energy delivery elements include electrodes.

8. The medical device of claim 1, wherein the one or more energy delivery elements are coupled to an ablation energy source.

9. The medical device of claim 1, wherein the one or more energy delivery elements are coupled to an electrical activity processing system.

10. A medical device, comprising:
an expandable-collapsible element slidably disposed within a catheter lumen, wherein the expandable-collapsible element includes a balloon configured to move between a deflated configuration and an inflated configuration; and
one or more energy delivery elements distributed along only one side of the balloon.

11. The medical device of claim 10, further comprising a vent tube passing through a center portion of the balloon.

12. The medical device of claim 10, wherein the one or more energy delivery elements include electrodes.

13. The medical device of claim 10, wherein the one or more energy delivery elements are coupled to an ablation energy source.

14. The medical device of claim 10, wherein the one or more energy delivery elements are coupled to an electrical activity processing system.

15. The medical device of claim 12, wherein the electrodes are configured to act as one or more of the following: sensing electrode, mapping electrode, stimulating electrode and ablation electrode.

16. A method, comprising:
introducing an expandable-collapsible element into a body lumen, the expandable-collapsible element comprising:
a framework formed from a plurality of longitudinal members, wherein the plurality of longitudinal members include regions of varying flexibility and stiffness configured to expand into a curved shape when not disposed within the catheter lumen; and
one or more energy delivery elements carried about an outer surface of at least one of the longitudinal members;
moving the expandable-collapsible element to an expanded configuration such that one or more of the energy delivery elements contact a tissue of the body lumen; and
delivering ablation energy from at least one of the energy delivery elements to the tissue of the body lumen.

17. The medical device of claim 16, wherein the energy delivery elements are carried about the longitudinal members on only one side of the framework.

18. The medical device of claim 16, wherein at least some of the longitudinal members are connected to each other by one or more cross-pieces.

19. The medical device of claim 18, wherein the longitudinal members and one or more cross-pieces form a mesh-like structure.

20. The medical device of claim 18, wherein the longitudinal members and one or more cross-pieces form a basket structure.
